(19) United States
(12) Patent Application Publication
(10) Pub. No.: US 2026/0208158 A1
(43) Pub. Date: Jul. 23, 2026

(54) PROCESS FOR THE PRODUCTION OF SUPERABSORBENTS

(71) Applicant: BASF SE, Ludwigshafen Am Rhein (DE)

(72) Inventors: Dieter SCHILLER, Ludwigshafen am Rhein (DE); Ivanilton Dos Reis SOUZA, Camacari (BR); Helvio VIEIRA AMORIM, Camacari (BR); Francisco Assis DA SILVA JUNIOR, Saint-Aubin-les-Elbeuf (FR)

(73) Assignee: BASF SE, Ludwigshafen Am Rhein (DE)

(21) Appl. No.: 19/145,292

(22) PCT Filed: Dec. 20, 2023
(86) PCT No.: PCT/EP2023/086834

§ 371 Date: Jul. 2, 2025

(30) Foreign Application Priority Data
Jan. 5, 2023 (EP) 23150381.4

(51) Int. Cl. B01J 20/26; A61L 15/60; B01J 20/28; B01J 20/30; C08F 220/06

(52) U.S. Cl. CPC B01J 20/267; A61L 15/60; B01J 20/28047; B01J 20/3007; B01J 20/3021; B01J 20/3078; B01J 20/3085; C08F 220/06; C08F 2810/20

(57) ABSTRACT

The present invention relates to a process for producing superabsorbents, wherein a monomer solution M is cooled by a heat exchanger W, the neutralization is interrupted to clean the heat exchanger W, and the heat exchanger W is emptied, filled with water or an aqueous solution and blow-dried.

The present invention relates to a process for producing superabsorbents, wherein a monomer solution M is cooled by means of a heat exchanger W, the neutralization is stopped for cleaning of the heat exchanger W, and the heat exchanger W is emptied, filled with water or an aqueous solution and cleared by blowing.

Superabsorbents are used to produce diapers, tampons, sanitary napkins and other hygiene articles, but also as water-retaining agents in market gardening. Superabsorbents are also referred to as water-absorbing polymers.

The production of superabsorbents is described in the monograph “Modern Superabsorbent Polymer Technology”, F. L. Buchholz and A. T. Graham, Wiley-VCH, 1998, pages 71 to 103.

In order to improve performance properties, for example gel bed permeability (GBP) and absorption under a pressure of 49.2 g/cm² (AUL0.7 psi), superabsorbent particles are generally surface postcrosslinked. This increases the level of crosslinking of the particle surface, which can at least partly decouple the absorption under a pressure of 49.2 g/cm² (AUL0.7 psi) and the centrifuge retention capacity (CRC). This surface postcrosslinking can be performed in the aqueous gel phase. Preferably, however, polymer particles (base polymer), having been dried, ground and sieved off, are surface coated with a surface postcrosslinker and thermally surface postcrosslinked. Crosslinkers suitable for that purpose are compounds which can form covalent bonds with at least two carboxylate groups of the polymer particles.

WO 2007/028751 A1 relates to a neutralization process.

It was an object of the present invention to provide an improved process for producing superabsorbent particles, in particular for easier cleaning of the heat exchangers used.

The object was achieved by a process for producing superabsorbents by at least partly neutralizing at least one ethylenically unsaturated monomer bearing acid groups with an aqueous base, cooling the resultant aqueous monomer solution M by means of a heat exchanger W, adding at least one crosslinker and at least one initiator to the aqueous monomer solution M, then polymerizing the aqueous monomer solution M to a polymer gel, optionally extruding the polymer gel, drying the polymer gel, and comminuting, classifying and optionally thermally surface postcrosslinking the dried polymer gel, wherein the neutralization is stopped for cleaning of the heat exchanger W, and the heat exchanger W is emptied, filled with water or an aqueous solution and cleared by blowing.

The heat exchanger W is preferably filled at least twice, for example twice, three times or four times, with water or an aqueous solution and cleared by blowing. The water or the aqueous solution preferably comprises essentially no polyvalent metal ions, in particular essentially no Ca²⁺ ions and/or Mg²⁺ ions.

In a preferred embodiment, the heat exchanger W is additionally filled with an alkaline solution, heated and emptied. The heat exchanger W is preferably filled at least twice, for example twice, three times or four times, with an alkaline solution, heated and emptied. The heat exchanger W may be emptied beforehand, filled with water or an aqueous solution, and cleared by blowing. Before that, the heat exchanger W is preferably filled at least twice, for example twice, three times or four times, with water or an aqueous solution and cleared by blowing.

The pH of the alkaline solution is preferably greater than 10, more preferably greater than 12, most preferably greater than 14. An example of a suitable alkaline solution is sodium hydroxide solution. The sodium hydroxide content in the sodium hydroxide solution is preferably at least 10% by weight, more preferably at least 25% by weight, most preferably at least 40% by weight.

The alkaline solution is heated in the heat exchanger W to a temperature of preferably at least 30° C., more preferably at least 45° C., most preferably at least 60° C. The alkaline solution is heated in the heat exchanger W for preferably at least 5 minutes, more preferably at least 15 minutes, most preferably at least 25 minutes.

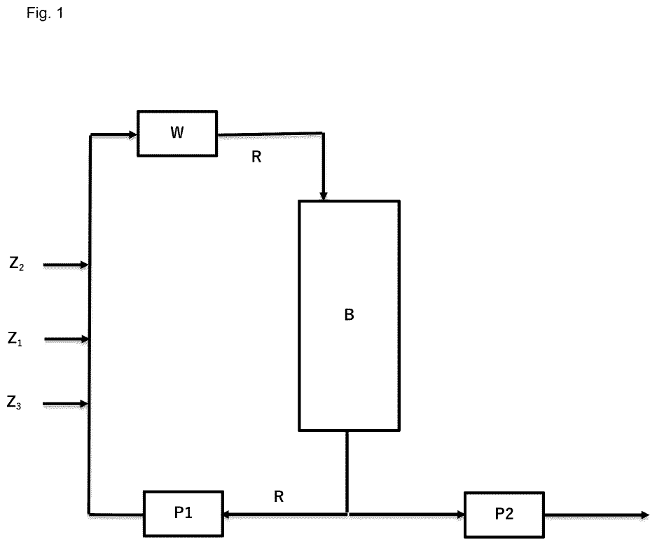

FIG. 1 shows an example of a neutralization process. The reference symbols are defined as follows:
W heat exchanger
R ring conduit
P1 pump in the ring conduit
P2 pump to the polymerization reactor
B (buffer) vessel
Z₁ feed
Z₂ feed
Z₃ feed

The present invention is based on the finding that the heat exchanger W can be cleaned in a simple manner and often without stopping the polymerization. It is possible to dispense with complex mechanical cleaning.

The heat exchangers W used in accordance with the invention are indirect heat exchangers and are also referred to as recuperators. There are plate heat exchangers, shell and tube heat exchangers, jacketed tube heat exchangers and mixed forms thereof. The heat exchangers W used are not subject to any restriction. Heat exchangers W that are preferred in accordance with the invention are plate heat exchangers. A plate heat exchanger consists of parallel plates, where the interspaces are alternately occupied by one medium and another. A spiral heat exchanger is a special form of plate heat exchanger, wherein a spiral-wound panel is used rather than two-dimensional plates.

The monomer solution M and the cooling medium may be conducted in countercurrent, cocurrent, crosscurrent or cross-countercurrent. Preference is given in accordance with the invention to countercurrent heat exchangers. In a countercurrent heat exchanger, the substances are guided such that they flow past one another in opposite directions. In the ideal case, the temperatures of the streams of matter are exchanged, meaning that the originally cold medium reaches the temperature of the originally hot medium and vice versa. In practice, however, complete exchange of temperatures is impossible.

The production of the superabsorbents is described in detail hereinafter:

The superabsorbents are produced by polymerizing a monomer solution and are typically water-insoluble.

The ethylenically unsaturated monomers bearing acid groups are preferably water-soluble, i.e. their solubility in water at 23° C. is typically at least 1 g/100 g of water, preferably at least 5 g/100 g of water, more preferably at least 25 g/100 g of water and most preferably at least 35 g/100 g of water.

Suitable monomers are, for example, ethylenically unsaturated carboxylic acids, such as acrylic acid, methacrylic acid and itaconic acid. Particularly preferred monomers are acrylic acid and methacrylic acid. Very particular preference is given to acrylic acid.

The ethylenically unsaturated monomers bearing acid groups have typically been partly neutralized. The neutralization is conducted at the monomer stage. This is typically accomplished by mixing in the neutralizing agent as an aqueous solution or else preferably as a solid. The degree of neutralization is preferably from 40 to 85 mol %, more preferably from 50 to 80 mol % and most preferably from 60 to 75 mol %, for which the customary neutralizing agents can be used, preferably alkali metal hydroxides, alkali metal oxides, alkali metal carbonates or alkali metal hydrogen-carbonates and also mixtures thereof. Instead of alkali metal salts, it is also possible to use ammonium salts. Particularly preferred alkali metals are sodium and potassium, but very particular preference is given to sodium hydroxide, sodium carbonate or sodium hydrogencarbonate and also mixtures thereof, especially sodium hydroxide.

The monomers typically comprise polymerization inhibitors, preferably hydroquinone monoethers, as storage stabilizers.

Suitable crosslinkers are compounds having at least two groups suitable for crosslinking. Such groups are, for example, ethylenically unsaturated groups which can be polymerized free-radically into the polymer chain, and functional groups which can form covalent bonds with the acid groups of the monomer. In addition, polyvalent metal salts which can form coordinate bonds with at least two acid groups of the monomer are also suitable as crosslinkers.

Suitable crosslinkers are, for example, ethylene glycol dimethacrylate, diethylene glycol diacrylate, polyethylene glycol diacrylate, allyl methacrylate, trimethylolpropane triacrylate, triallylamine, tetraallylammonium chloride, tetraallyloxyethane, as described in EP 0 530 438A1 , di- and tri-acrylates, as described in EP 0 547 847A1, EP 0 559 476A1, EP 0 632 068A 1, WO 93/21237 A1, WO 03/104299A1, WO 03/104300A1, WO 03/104301 A1 and DE 103 31 450A1, mixed acrylates which, as well as acrylate groups, comprise further ethylenically unsaturated groups, as described in DE 103 31 456 A1 and DE 103 55 401 A1 , or crosslinker mixtures, as described, for example, in DE 195 43 368 A1, DE 196 46 484 A1, WO 90/15830 A1 and WO 02/032962A2.

The amount of crosslinker is preferably 0.05% to 1.5% by weight, more preferably 0.1% to 1% by weight and most preferably 0.15% to 0.6% by weight, calculated in each case on the basis of the total amount of monomer used. With rising crosslinker content, the centrifuge retention capacity (CRC) falls and the absorption under a pressure of 21.0 g/cm² (AUL0.3 psi) passes through a maximum.

Initiators used may be all compounds which generate free radicals under the polymerization conditions, for example thermal initiators, redox initiators or photoinitiators. Suitable redox initiators are sodium peroxodisulfate/ascorbic acid, hydrogen peroxide/ascorbic acid, sodium peroxodisulfate/sodium bisulfite and hydrogen peroxide/sodium bisulfite. Preference is given to using mixtures of thermal initiators and redox initiators, such as sodium peroxodisulfate/hydrogen peroxide/ascorbic acid. The reducing component used is preferably the disodium salt of 2-hydroxy-2-sulfonatoacetic acid or a mixture of the sodium salt of 2-hydroxy-2-sulfinatoacetic acid, the disodium salt of 2-hydroxy-2-sulfonatoacetic acid and sodium bisulfite. Such mixtures are obtainable as Bruggolite® FF6 and Bruggolite® FF7 (Brüggemann Chemicals; Heilbronn; Germany).

The water content of the monomer solution M is preferably from 40% to 75% by weight, more preferably from 45% to 70% by weight and most preferably from 50% to 65% by weight. As the water content rises, the energy expenditure in the subsequent drying rises and, as the water content falls, the heat of polymerization can only be removed inadequately.

The temperature of the monomer solution M is preferably from 10 to 90° C., particularly preferably from 20 to 70° C., very particularly preferably from 30 to 50° C.

For optimal action, the preferred polymerization inhibitors require dissolved oxygen. The monomer solution can therefore be freed of dissolved oxygen before the polymerization by inertization, i.e. flowing an inert gas through, preferably nitrogen or carbon dioxide. The oxygen content of the monomer solution is preferably lowered before the polymerization to less than 1 ppm by weight, more preferably to less than 0.5 ppm by weight, most preferably to less than 0.1 ppm by weight.

Suitable reactors for the polymerization are, for example, kneading reactors or belt reactors. In the kneader, the polymer gel formed in the polymerization of an aqueous monomer solution or suspension is comminuted continuously by, for example, contrarotatory stirrer shafts, as described in WO 2001/038402A1. Polymerization on the belt is described, for example, in DE 38 25 366 A1 and U.S. Pat. No. 6,241,928. Polymerization in a belt reactor forms a polymer gel which has to be comminuted, for example in an extruder or kneader.

To improve the drying properties, the comminuted polymer gel obtained by means of a kneader can additionally be extruded.

The polymer gel is then typically dried with an air circulation belt drier until the residual moisture content is preferably 0.5% to 10% by weight, more preferably 1% to 7% by weight and most preferably 2% to 5% by weight, the residual moisture content being determined by EDANA recommended test method No. WSP 230.2-05 “Mass Loss Upon Heating”. In the case of too high a residual moisture content, the dried polymer gel has too low a glass transition temperature T_{g} and can be processed further only with difficulty. In the case of too low a residual moisture content, the dried polymer gel is too brittle and, in the subsequent comminution steps, undesirably large amounts of polymer particles with an excessively low particle size are obtained (“fines”). The solids content of the polymer gel before the drying is preferably from 25% to 90% by weight, more preferably from 35% to 70% by weight, most preferably from 40% to 60% by weight. Subsequently, the dried polymer gel is crushed and optionally coarsely comminuted.

Thereafter, the dried polymer gel is typically ground and classified, and the apparatus used for grinding may typically be single- or multistage roll mills, preferably two- or three-stage roll mills, pin mills, hammer mills or vibratory mills.

The average particle size of the polymer particles removed as the product fraction is preferably from 150 to 850 μm, more preferably from 250 to 600 μm, very particularly from 300 to 500 μm. The average particle size of the product fraction may be determined by means of EDANA recommended test method No. WSP 220.2 (05) “Particle Size Distribution”, where the proportions by mass of the screen fractions are plotted in cumulative form and the average particle size is determined graphically. The average particle size here is the value of the mesh size which arises for a cumulative 50% by weight.

To further improve the properties, the polymer particles can be thermally surface postcrosslinked. Suitable surface postcrosslinkers are compounds which comprise groups which can form covalent bonds with at least two carboxylate groups of the polymer particles. Suitable compounds are, for example, polyfunctional amines, polyfunctional amido amines, polyfunctional epoxides, as described in EP 0 083 022 A2, EP 0 543 303 A1 and EP 0 937 736 A2, di- or polyfunctional alcohols, as described in DE 33 14 019 A1, DE 35 23 617 A1 and EP 0 450 922 A2, or β-hydroxyalkylamides, as described in DE 102 04 938 A1 and U.S. Pat. No. 6,239,230.

The amount of surface postcrosslinker is preferably 0.001% to 2% by weight, more preferably 0.01% to 1% by weight and most preferably 0.03% to 0.7% by weight, based in each case on the polymer particles.

In a preferred embodiment of the present invention, polyvalent cations are applied to the particle surface in addition to the surface postcrosslinkers.

The polyvalent cations usable in the process of the invention are, for example, divalent cations such as the cations of zinc, magnesium, calcium and strontium, trivalent cations such as the cations of aluminum, iron, chromium, rare earths and manganese, tetravalent cations such as the cations of titanium and zirconium. Possible counterions are chloride, bromide, hydroxide, sulfate, hydrogensulfate, carbonate, hydrogencarbonate, nitrate, phosphate, hydrogenphosphate, dihydrogenphosphate and carboxylate, such as acetate and lactate. Aluminum hydroxide, aluminum sulfate and aluminum lactate are preferred.

The amount of polyvalent cation used is, for example, 0.001% to 1.5% by weight, preferably 0.005% to 1% by weight and more preferably 0.02% to 0.8% by weight, based in each case on the polymer.

The surface postcrosslinking is typically performed in such a way that a solution of the surface postcrosslinker is sprayed onto the dried polymer particles. After the spray application, the surface postcrosslinker-coated polymer particles are subjected to thermal treatment.

The spray application of a solution of the surface postcrosslinker is preferably performed in mixers with moving mixing tools, such as screw mixers, disk mixers and paddle mixers. Particular preference is given to horizontal mixers such as paddle mixers, very particular preference to vertical mixers. The distinction between horizontal mixers and vertical mixers is made by the mounting of the mixing shaft, i.e. horizontal mixers have a horizontally mounted mixing shaft and vertical mixers have a vertically mounted mixing shaft. Suitable mixers are, for example, horizontal Pflugschar® plowshare mixers (Gebr. Lödige Maschinenbau GmbH; Paderborn; Germany), Vrieco-Nauta continuous mixers (Hosokawa Micron BV; Doetinchem; the Netherlands), Processall Mixmill mixers (Processall Incorporated; Cincinnati; USA) and Schugi Flexomix® (Hosokawa Micron BV; Doetinchem; the Netherlands). However, it is also possible to spray on the surface postcrosslinker solution in a fluidized bed.

The surface postcrosslinkers are typically used in the form of an aqueous solution. The penetration depth of the surface postcrosslinker into the polymer particles can be adjusted via the content of nonaqueous solvent and total amount of solvent.

The thermal treatment is preferably conducted in contact driers, more preferably paddle driers, most preferably disk driers. Suitable driers are, for example, Hosokawa Bepex® Horizontal Paddle Dryer (Hosokawa Micron GmbH; Leingarten; Germany), Hosokawa Bepex® Disk Dryer (Hosokawa Micron GmbH; Leingarten; Germany), Holo-Flite® driers (Metso Minerals Industries Inc.; Danville; USA) and Nara Paddle Dryer (NARA Machinery Europe; Frechen; Germany). Moreover, fluidized bed driers may also be used.

The surface postcrosslinking can be effected in the mixer itself, by heating the jacket or blowing in warm air. Equally suitable is a downstream drier, for example a tray drier, a rotary tube oven or a heatable screw. It is particularly advantageous to effect mixing and thermal surface postcrosslinking in a fluidized bed drier.

Preferred reaction temperatures are in the range of 100 to 250° C., preferably 110 to 220° C., more preferably 120 to 210° C., most preferably 130 to 200° C. The preferred dwell time at this temperature is preferably at least 10 minutes, more preferably at least 20 minutes, most preferably at least 30 minutes, and typically at most 60 minutes.

Subsequently, the surface postcrosslinked polymer particles can be classified again, with excessively small and/or excessively large polymer particles being removed and recycled into the process.

To further improve the properties, the surface postcrosslinked polymer particles can be coated or remoisturized.

Remoisturizing is preferably performed at 30 to 80° C., more preferably at 35 to 70° C., most preferably at 40 to 60° C. At excessively low temperatures the polymer particles tend to form lumps, and at higher temperatures water already evaporates to a noticeable degree. The amount of water used for remoisturizing is preferably from 1% to 10% by weight, more preferably from 2% to 8% by weight and most preferably from 3% to 5% by weight. The remoisturizing increases the mechanical stability of the polymer particles and reduces their tendency to static charging. Remoisturizing is advantageously performed in a cooler after the thermal surface postcrosslinking.

Suitable coatings for improving the swell rate and the gel bed permeability (GBP) are, for example, inorganic inert substances, such as water-insoluble metal salts, organic polymers, cationic polymers and di- or polyvalent metal cations. Suitable coatings for dust binding are, for example, polyols. Suitable coatings for counteracting the undesired caking tendency of the polymer particles are, for example, fumed silica, such as Aerosil® 200, precipitated silica, such as Sipernat® D17, and surfactants, such as Span® 20.

### EXAMPLES

### Example 1 (Inventive)

By continuously mixing deionized water, 50% by weight sodium hydroxide solution and acrylic acid, a monomer solution M was prepared (see FIG. 1) such that the degree of neutralization corresponded to 72.0 mol %. The water content of the monomer solution M was 57.0% by weight.

In the ring conduit R, partly neutralized acrylic acid was circulated by means of the pump P1 via the heat exchanger W and the vessel B. Water was metered in via the feed Z₁, sodium hydroxide solution via the feed Z₃, and acrylic acid via the feed Z₂. By means of the pump P2, the monomer solution M was conveyed into the polymerization reactor.

The heat exchanger W used was a plate heat exchanger having an area of 178 m².

The crosslinker used was 3-tuply ethoxylated glyceryl triacrylate (purity about 85% by weight). The amount used was 0.95 kg per t of monomer solution M. Additionally added to the monomer solution M were 2.64 kg of polyethylene glycol (polyethylene glycol having an average molar mass of 4000 g/mol) and 9.69 kg of the disodium salt of 1-hydroxyethylidene-1,1′-diphosphonic acid (Cublen®K9012GR), in each case per t of monomer solution M.

The free-radical polymerization was initiated using, per t of monomer solution M, 1.13 kg of a 0.25% by weight aqueous hydrogen peroxide solution, 4.70 kg of a 15% by weight aqueous sodium peroxodisulfate solution and 1.06 kg of a 1% by weight aqueous ascorbic acid solution.

The monomer solution M was introduced into a List Contikneter continuous kneader reactor with a capacity of 6.3 m³ (LIST AG, Arisdorf, Switzerland). The throughput of the monomer solution M was about 22 t/h. The reaction solution had a feed temperature of 23.5° C.

Between the addition point for the crosslinker and the addition sites for the hydrogen peroxide and sodium peroxodisulfate solutions, the monomer solution M was inertized with nitrogen. Ascorbic acid was metered directly into the reactor.

After about 50% of the dwell time, an additional about 1000 kg/h of polymer particles obtained in the production process by comminution and classification, having a particle size of less than 150 μm, was metered into the reactor. The residence time of the reaction mixture in the reactor was about 15 minutes.

The polymer gel obtained was applied to the conveyor belt of an air circulation belt drier by means of an oscillating conveyor belt. The air circulation belt drier had a length of 48 m. The conveyor belt of the air circulation belt drier had an effective width of 4.4 m. The aqueous polymer gel on the air circulation belt drier was subjected to a continuous flow of air/gas mixture and dried.

The dried polymer gel was comminuted by means of a three-stage roll mill and sieved off to a particle size of 150 to 700 μm. Polymer particles having a particle size of less than 150 μm were separated off. Polymer particles having a particle size of greater than 700 μm were recycled into the comminution. Polymer particles having a particle size in the range from 150 to 700 μm were thermally surface postcrosslinked.

The polymer particles were coated with a surface postcrosslinker solution in a Schugi Flexomix® (Hosokawa Micron B.V., Doetinchem, the Netherlands) and then dried in a NARA Paddle Dryer (GMF Gouda, Waddinxveen, the Netherlands) at 192.5° C. for 45 minutes.

The following amounts were metered into the Schugi Flexomix®:
9.5 t/h of polymer particles 488.4 kg/h of surface postcrosslinker solution

The surface postcrosslinker solution comprised 1.36% by weight of 2-hydroxyethyl-2-oxazolidone, 1.36% by weight of propane- 1,3-diol, 4.28% by weight of aluminum lactate, 54.04% by weight of water, 0.05% by weight of sorbitan monolaurate (Span®20) and 38.91% by weight of isopropanol.

After drying, the surface postcrosslinked polymer particles were cooled down to about 60° C. in a NARA Paddle-Cooler (GMF Gouda, Waddinxveen, the Netherlands). The surface postcrosslinked polymer particles were coated here with 285 kg/h of water, 1.67 kg/h of a 50% by weight aqueous polyethylene glycol solution (polyethylene glycol having an average molar mass of 400 g/mol), 23.75 kg/h of a 1% aqueous sorbitan monolaurate solution and 9.5 kg/h of silicon dioxide (Sipernat®22S).

The delivery rate of the pump P 1 in regular operation is 300 t/h. After a while, the delivery rate of the pump P 1 in the neutralization fell below 200 t/h owing to soiling in the plate heat exchanger W.

With continuing polymerization, feed and drain for the monomer solution in the heat exchanger W were closed. The heat exchanger W was emptied via a separate conduit having an internal diameter of about 7.5 cm. At the same time, the heat exchanger W was ventilated via a further conduit having an internal diameter of about 2.5 cm.

Subsequently, the heat exchanger W was filled with demineralized water within 2 minutes. After about a further 2 minutes, the demineralized water was blown out using compressed air. Filling with demineralized water and clearing by blowing were repeated two to four times.

Thereafter, feed and drain for the monomer solution in the heat exchanger W were opened again. The time taken was about 40 minutes in total.

In the cases where the above cleaning did not lead to a clear improvement, the polymerization was stopped and the neutralization was emptied. Thereafter, feed and drain for the monomer solution in the heat exchanger W were closed.

Subsequently, the heat exchanger W was filled with demineralized water within 2 minutes. After about a further 2 minutes, the demineralized water was blown out using compressed air. Filling with demineralized water and clearing by blowing were repeated three times.

Thereafter, the heat exchanger W was filled with 50% by weight sodium hydroxide solution within about 3 minutes, heated to about 70° C. for about 30 minutes and emptied. The filling with sodium hydroxide solution, the heating and the emptying were likewise repeated three times.

Subsequently, the heat exchanger W was filled with demineralized water within 2 minutes. After about a further 2 minutes, the demineralized water was blown out using compressed air. Filling with demineralized water and clearing by blowing were repeated three times.

Thereafter, neutralization and polymerization were restarted. The time taken was about 6 hours in total.

### Example 2 (Not According to the Invention)

The procedure was as in example 1. The heat exchanger W was disassembled and mechanically cleaned in a complex manner.

Thereafter, neutralization and polymerization were restarted. The time taken was about 12 hours in total.

1: A process for producing superabsorbents by, comprising:
at least partly neutralizing at least one ethylenically unsaturated monomer bearing acid groups with an aqueous base;
cooling a resultant aqueous monomer solution M with a heat exchanger W;
adding at least one crosslinker and at least one initiator to the aqueous monomer solution M;
then polymerizing the aqueous monomer solution M to a polymer gel;
optionally extruding the polymer gel;
drying the polymer gel; and
comminuting, classifying and optionally thermally surface postcrosslinking the dried polymer gel;
wherein:
the neutralization is stopped for cleaning of the heat exchanger W; and
the heat exchanger W is emptied, filled with water or an aqueous solution and cleared by blowing.

2: The process according to claim 1, wherein the heat exchanger W is at least twice filled with water or an aqueous solution and cleared by blowing.

3: The process according to claim 1, wherein the heat exchanger W is additionally filled with an alkaline solution, heated, and emptied.

4: The process according to claim 3, wherein the heat exchanger W is at least twice filled with an alkaline solution, heated, and emptied.

5: The process according to claim 3, wherein the alkaline solution a sodium hydroxide solution.

6: The process according to claim 5, wherein the sodium hydroxide solution has a sodium hydroxide content of at least 40% by weight.

7: The process according to claim 3, wherein the alkaline solution is heated to at least 60° C. in the heat exchanger W.

8: The process according to claim 3, wherein the alkaline solution is heated for at least 25 minutes in the heat exchanger W.

9: The process according to claim 1, wherein a plate heat exchanger is used as heat exchanger W.

10: The process according to claim 1, wherein the ethylenically unsaturated monomer bearing acid groups used is an ethylenically unsaturated carboxylic acid.

11: The process according to claim 1, wherein the ethylenically unsaturated monomer bearing acid groups used is acrylic acid.

12: The process according to claim 1, wherein the base comprises at least one selected from the group consisting of an alkali metal hydroxide, an alkali metal oxide, an alkali metal hydrogencarbonate, and an alkali metal carbonate.

13: The process according to claim 1, wherein the base is sodium hydroxide.

14: The process according to claim 1, wherein the ethylenically unsaturated monomer bearing acid groups is neutralized to an extent of 60 to 75 mol %.

15: The process according to claim 1, wherein a water content of the monomer solution M is 50% to 65% by weight.
